(19) United States
(12) Patent Application Publication
(10) Pub. No.: US 2008/0312648 A1
(43) Pub. Date: Dec. 18, 2008

(54) Fat removal and sculpting device

(76) Inventor: Darion Peterson, Boulder, CO (US)

Correspondence Address: Tyco Healthcare Group LP, 60 MIDDLETOWN AVENUE, NORTH HAVEN, CT, 06473, US

(21) Appl. No.: 11/811,794

(22) Filed: Jun. 12, 2007

(51) Int. Cl. A61B 18/14

(52) U.S. Cl. 606/41

(57) ABSTRACT

A device for removing and/or sculpting tissue includes a substantially planar pad and an array of needles extending distally from the pad. Each of the needles is configured for insertion into body tissue, and each of the needles may be configured to couple to a source of electrosurgical energy.

### BACKGROUND

1. Technical Field

The present disclosure relates to the removal of unwanted fat from the body and, more particularly, to a less evasive or invasive device and method of performing the same.

2. Background of Related Art

When traditional methods of body shaping and sculpting, such as diet and exercise, require too much time and effort or are just not enough to loose that unwanted fat, individuals turn to plastic surgeons for assistance. Excess subcutaneous fat, including cellulite, in addition to being an increased risk factor, may cause unevenness or rippling of the skin. Plastic surgeons are able to effectively remove or sculpt subcutaneous fat using a technique commonly referred to as liposuction. Liposuction is a type of cosmetic surgery where undesirable accumulations of body fat are removed by suction. Liposuction is seen by many as a quick and easy, albeit painful, way to tone up.

Conventional liposuction is performed using a cannula connected to an external source of suction. An incision is made in the area of the targeted subcutaneous fat desired to be removed, the cannula is inserted into the area, and the suction is started. The cannula is moved back and forth to separate the fat from the surrounding tissue. Unfortunately, the fat is relatively difficult to separate from the tissue. Such separation sometimes causes excessive bleeding.

Additionally, the cannula has a tendency to clog during use, therefore, it is difficult to keep the operation going without stopping to clean out the cannula. The surgeon normally attempts to compensate for this problem by rapidly moving the cannula within the cavity. Periodically he may even withdrawing cannula from the within the patient to allow the fat to move therethrough. To further complicate the procedure, the surgeon must be careful not to allow the cannula or suction from the cannula to remove or injure any desirable tissues, such as muscle, blood vessels, skin, other subcutaneous tissues, and the like. Although conventional liposuction has proven successful for the removal of fat, the evasiveness of the procedure leaves much to be desired.

### SUMMARY

According to one embodiment of the disclosure, a device for removing and/or sculpting tissue includes a substantially planar pad and an array of needles extending distally from the pad. Each of the needles is configured for insertion into body tissue, and each of the needles may be configured to couple to a source of electrosurgical energy.

Each of the needles may define at least one lumen configured for fluid communication with an aspiration/suction source. Each of the needles may be configured for independent activation. The array of needles may be configured for simultaneous activation and the at least one lumen is configured for suction.

The pad may include a first layer and a second layer. The first layer may be configured to retain the needles. The first layer may also be extended relative to the second layer.

According to another embodiment of the disclosure, a method of removing and/or sculpting fat includes the steps of providing a fat removal device having an array of needles extending distally from a pad, each of the needles coupled to a source of electrosurgical energy, aligning the fat removal device with a target tissue, penetrating the array of needles into the target tissue, and activating the source of electrosurgical energy to deliver electrosurgical energy through the array of needles.

The method may further include removing fat from the target tissue via an aspiration/fluid source coupled to the needles and providing fluid through the needles.

The method may further include providing a first power level of electrosurgical energy to a first set of the array of needles and a second power level of electrosurgical energy to a second set of the array of needles. The method may further include independently activating at least some of the needles.

### BRIEF DESCRIPTION OF THE DRAWINGS

The foregoing summary, as well as the following detailed description will be better understood when read in conjunction with the appended figures. For the purpose of illustrating the present disclosure, a preferred embodiment is shown. It is understood, however, that the present disclosure is not limited to the precise arrangement and instrumentalities shown.

FIG. 1 is a perspective view of a fat removal/sculpting device in accordance with an embodiment of the present disclosure including a base and an array of needles;

FIG. 2 is an enlarged cross-sectional side view of the base and a needle of the fat removal/sculpting device of FIG. 1;

FIG. 3 is a multiple cross-sectional top view of the fat removal/sculpting device of FIGS. 1-3;

FIG. 4 is a perspective view of a fat removal/sculpting device according to another embodiment of the present disclosure, in a fully extend condition;

FIG. 5 is the fat removal/sculpting device of FIG. 4 in a fully retracted condition; and

FIG. 6 is a cross-sectional side view of the base and a needle of the fat removal/sculpting device of FIG. 5.

### DETAILED DESCRIPTION OF PREFERRED EMBODIMENTS

The foregoing summary, as well as the following detailed description will be better understood when read in conjunction with the appended figures. For the purpose of illustrating the present disclosure, various embodiments are shown. It is understood, however, that the present disclosure is not limited to the precise arrangement and instrumentalities shown.

As shown in the drawings and described throughout the following description, as is traditional when referring to relative positioning on an object, the term “proximal” refers to the end of the apparatus that is closer to the user and the term “distal” refers to the end of the apparatus that is further from the user.

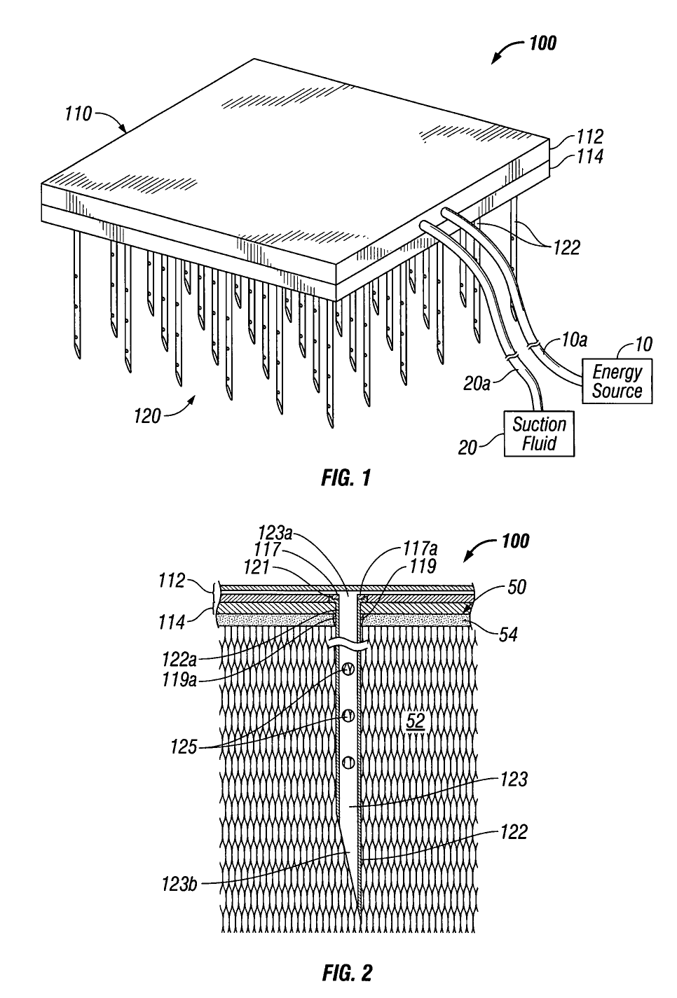

Referring to FIGS. 1-3, an illustrative embodiment of the presently disclosed fat removal device is shown therein and is generally designated as device 100. Fat removal device 100 includes a substantially planar base or pad 110 and an array 120 of needles 122 extending distally from a tissue contacting surface of base 110. Device 100 may be configured with base 110 of any size and includes an array 120 of needles 122 of any number, size, length, and configuration. As will be discussed below, needles 122 are preferably small enough to minimize scarring from insertion of needle array 120 through skin 54 (FIG. 2) of a patient. As will also be described below, device 100 may be configured to couple to an energy source 10 by a suitable wire 10a extending therefrom. Device 100 may further be configured for fluid communication with a suction and/or aspiration source 20 through a suitable tube or hose 20a extending therefrom.

Referring initially to FIGS. 1 and 2, base 110 forms a pad-like member including a first layer 112 and a second layer 114. Preferably, first and second layers 112, 114 are flexible and are able to conform to the contours of a patient's body. In some embodiments, first and second layers 112, 114 may be formed of plastic, polymer, cloth or other suitable substantially non-conductive material. Additionally, depending on the configuration of first and second layers 112, 114 and the engagement with needle array 120, first and second layers 112, 114 may further be formed of metal or other suitable conductive material. First and second layers 112, 114 may be formed from the same material or different materials. First layer 112 may be securely affixed to second layer 114 using any suitable known technique, including and not limited to adhesives, bonding, welding, mechanical fasteners, press fit coupling, etc. Alternatively, first layer 112 may be releasably secured to second layer 114 using any of the above listed techniques. In an alternate embodiment, first and second layers 112, 114 are integrally formed.

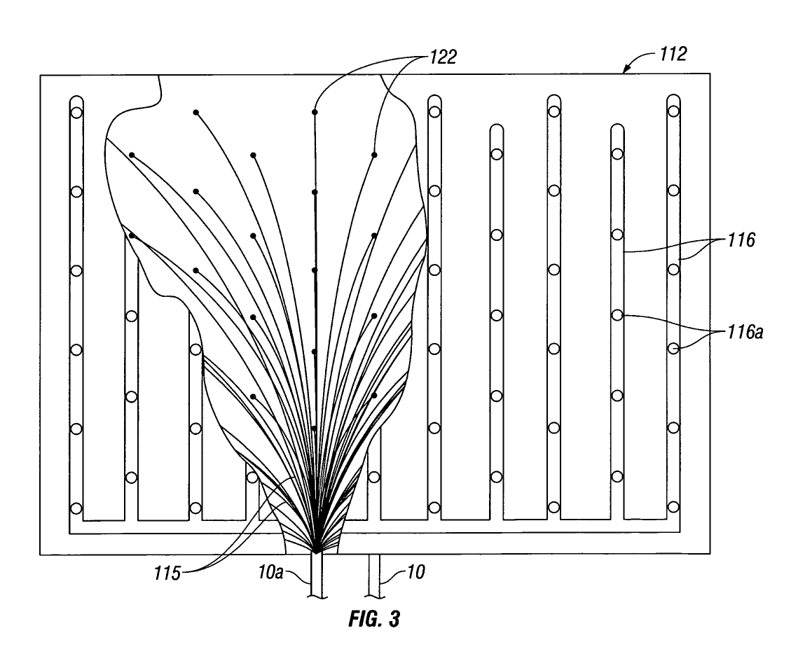

Turning now to FIG. 3, first layer 112 defines a plurality of channels 116 formed therein. First layer 112 further defines opening 116a positioned within channels 116. Openings 116a fluidly communicate needles 122 of needle array 120 with suction or fluid source 20 (FIG. 1). In this manner, channels 116 and openings 116a may communicate needles 120 with suction or fluid. Channels 116 may be formed within first layer 112, as shown (FIG. 2) or, in other embodiments, channels 116 may be formed in either first or second layer 112, 114, or may be formed between first and second layers 112, 114. Channels 116 may be arranged in vertical rows as shown, or, alternatively, channels 116 may be arranged in horizontal rows, vertical and horizontal rows, or as one large cavity fluidly communicating with openings 116a.

Still referring to FIG. 3, within either first or second layer 112, 114 device 100 includes connector cord 10a (FIG. 1) for coupling needle array 120 with source of energy 10. Device 100 includes connector wires 115 coupling needles 120 with energy source 10 via connector cord 10a. Alternatively, needles 120 may be coupled to energy source 10 using a conductive plate (not shown) secured with or between first and second layers 112, 114. The connector plate may be coupled to needle array 120.

Each needle 122 may be independently connected with energy source 10 such that each needle 122 may be individually activated apart from the activation of other needles 122 (i.e., independent activation). A single wire (not shown) may instead be used to connect each needle 122 together and to energy source 10. As will be discussed in more detail below, energy source 10 may provide AC, DC or RF currents, microwaves, ultrasound and the like to each needle 122. Needles 122 may be configured to be activated all together, individually or in any suitable pattern.

Referring back to FIG. 2, in the illustrated embodiment, first and second layer 112, 114 cooperate to form respective openings 117, 119 for receiving a proximal end 122a of each needle 122 therein. Alternatively, second layer 114 may be configured to completely receive each needle 122 therein without cooperation from first layer 112. Each needle 122 may be permanently affixed within first and/or second layers 112, 114. In an alternate embodiment, needle 122 may be releasably secured within first and second layers 112, 114. In this manner, any particular needle 122, or needle array 120, may be replaced as necessary. Needle 122 or needle array 120 may need to be changed when they become damaged or because the procedure being performed requires needles 120 of different dimensions.

With continued reference to FIG. 2, tab 117a extends from first layer 112 about opening 117 formed therein. Tab 117a is configured for matingly engaging proximal end 122a of each needle 122. As discussed above, proximal end 122a of each needle 122 may instead be completely received within second layer 114. As such, second layer 114 may include tabs 117a extending into openings 119 and configured to receive needle 122 in a manner similar to that described above.

With reference to FIG. 3, needles 122 are arranged in a linear or rectilinear pattern forming offset parallel rows. However, first and/or second layers 112, 114 may be configured to receive needles 122 in a variety of patterns. Preferably, needles 122 are arranged in a pattern specifically and suitably adapted for the procedure being performed. Needles 122 may be spaced sufficiently close to provide an enhanced effect in removing fat, but not so close as to interfere with the operation of the other needles 122. Alternatively, needles 122 may be configured to operate or cooperate with one another, e.g. as one set of needles 122 aspirate a fluid a second set of adjacent needles 122 may remove the aspiration fluid and tissue 52 from target site 50.

Second layer 114 may include spacers 119a (FIG. 2) extending distally from about openings 119. As will be discussed below, once needles 122 have been placed through the skin and into target tissue 52, needles 122 are energized. Spacers 119a prevent damage to skin 50 surrounding needle 122 as needle 122 is energized. Spacers 119a may be integrally formed with second layer 114. Alternatively, spacers 119a may be releasably engageable with second layer 114. Spacers 119a may be sharpened to assist in penetration of skin 50.

With continued reference to FIG. 2, each needle 122 forms a substantially tubular member having proximal end 122a and a distal end 122b. As described above, proximal end 122a of needle 122 is configured to be received within first and/or second layer 112, 114 of device 100. Proximal end 122a of needle 122 includes a flange 121 for matingly engaging tab 117a formed in first layer 112. Proximal end 123a of each needle 122 may be configured to be coupled to wire 115a (FIG. 3) or a conductive plate, as described above. In this manner, energy may be directed from energy source 10 through each needle 122 to targeted tissue 52. Distal end 122a of each needle 122 may form a sharpened point configured for piercing tissue. Each needle 122 may be formed of a standard length that may be cut to size. Alternatively, each needle 122 may be formed of various lengths such that cutting of needle 122 is not necessary. Each needle 122 may be of any suitable length and any suitable diameter.

In one embodiment, each needle 122 is of a diameter sufficiently small as to minimize or eliminate scarring. Needle 122 preferably defines a lumen 123 therethrough; however, each needle 122 may be solid. Lumen 123 includes proximal and distal ends 123a, 123b. Proximal end 123a of lumen 123 is configured for fluid communication with opening 116a formed in channel 116. Lumen 123 may include an open distal end 123, as shown. In an alternative embodiment, distal end 123b of lumen 123 may be closed.

Each needle 122 may further define one or more openings 125 in fluid communication with lumen 123. Openings 125 are formed along the length of each needle 122, in a side wall thereof, and are configured to permit discharge of fluid from within needle 122 and/or to provide suction for the removal of fluid and tissue. Openings 125 may be angled relative to a longitudinal axis of lumen 123 for directing the fluid or suction in a particular direction. Openings 125 of a first needle 122 in the array 120 of needles 122 may be positioned to interact with openings 125 of a second or adjacent needle 122. In this manner, openings 125 may work in combination or cooperation with one another to assist in removal of tissue and/or fluid from within the body or to aspirate or provide fluid thereto. Each of openings 125 may include a bevel or contour for preventing clogging thereof.

Needle array 120 of device 100 may be configured, as shown, in offset parallel rows. The configuration of needle array 120 may be specific to the particular site in which fat is to be removed and will vary from patient to patient. The configuration of needle array 120 may even vary from site to site within a patient. Additionally, the length of needles 122 may be varied depending on the site being targeted and the amount of tissue being removed. The length of each needle 122 within the array of needles 120 may be varied throughout device 100 depending on the application.

As discussed above, needles 122 may be coupled to a suction or aspiration source for either supplying fluid to or removing tissue and fluid from the target site. Connection of device 100 is through hose 20a (FIG. 1). Hose 20a may include one or more lumen(s) for selectively applying fluid, e.g., saline, to target tissue 52 or for providing suction to the same. Device 100 may be configured such that a portion of needle array 120 operates to discharge fluid while the remainder of needle array 120 are either inoperative or operate to remove the fluid and tissue from the target site. Alternatively, a first portion of needle array 120 may be configured to discharge a first fluid to the target tissue 52 and a second portion of needles 120 may be configured to discharge a second fluid to target tissue 52. The first and second fluids may interact to assist in the removal of fat from the target site 50.

In operation, and with reference to FIGS. 1 and 2, fat removal device 100 is preferably configured for a particular application. The surface area to be targeted, the depth of the tissue to be removed, the composition of the tissue, and the like, affect the configuration of fat removal device 100. Once the proper configuration has been determined and fat removal device 100 has been assembled, the array of needles 120 of device 100 may be inserted, percutaneously, through skin 54 of the target site 50 and into target tissue 52. Upon complete insertion of needles 122 through skin 54 and into target tissue 52, fat removal device 100 is ready for activation. Electrosurgical energy may be applied to needles 122 by energy source 10 as described above. The electrosurgical energy may pass through needles 122 and into the target tissue 52. The electrosurgical energy may instead cause heating of needles 122 or the fluid that may be passing therethrough. The electrosurgical energy provided to needles 122 will cause target tissue 52 to melt or emulsify. Aspiration and/or suction device 20 may then be used to supply fluid to and/or remove fluid from tissue 52 of target site 50 through needles 122. In other embodiments, the melted tissue may be left at target site 50 for reabsorption by the body. Once the desired amount of fat has been melted and/or removed, fat removal device 100 may be removed from target site 50. Preferably, the size and configuration of fat removal device 100 causes minimal, if any, scarring.

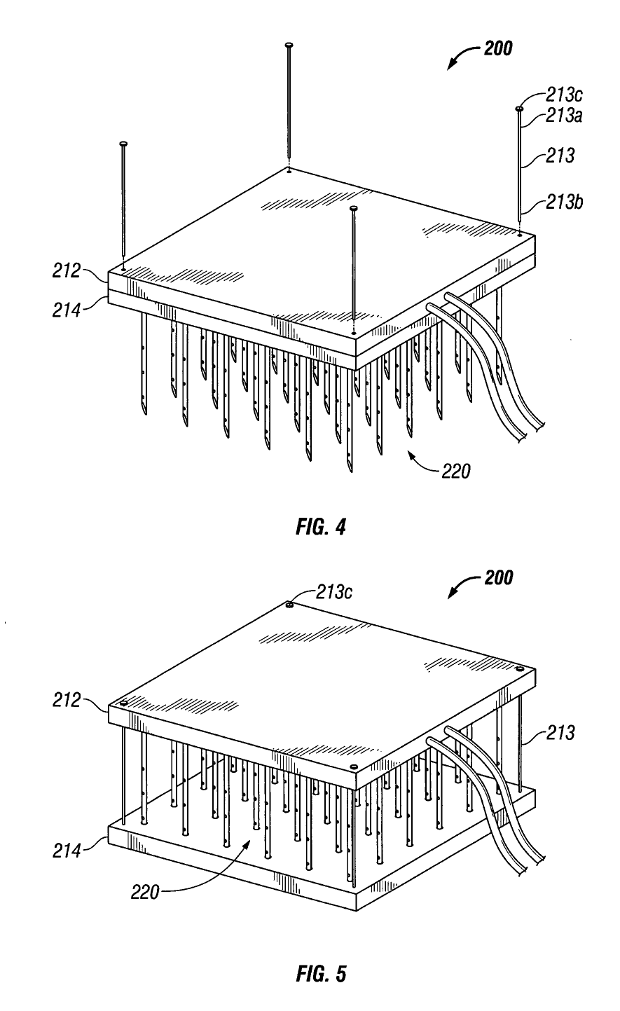

Turning now to FIGS. 4-6, an alternate embodiment of presently disclose fat removal system is shown generally as fat removal device 200. Referring initially to FIGS. 4 and 5, fat removal device 200 is substantially similar to fat removal device 100 and therefore, only the difference in construction and operation between devices 100, 200 will be discussed. Fat removal device 200 includes a substantial planar base or pad 210 and an array 220 of needles 222 extending distally from base 210. Base 210 includes first and second layers 212, 214. First and second layers 212, 214 may be selectively spaced from one another (FIG. 5) along guides 213 extending from second layer 214. Thus, in operation, fat removal device 200 may be placed into position on the skin of a patient prior to insertion of needles 222 therein. In this manner, fat removal device 200 may be properly aligned on the skin of the patient prior to insertion of needles 222, thereby minimizing any scarring or injury that may occur if needles 222 have to be withdrawn and reinserted into the patient. Alternatively, needles 222 may be inserted and retracted into the target tissue in a fashion similarly used in liposuction, whereby the needles tear the target tissue from surrounding tissue.

Guides 213 include proximal and distal ends 213a, 213b. Proximal end 213a forms a head 213c for preventing first layer 212 from sliding off of guide 213. Head 213c may further prevent withdrawal of needles 220 from within second layer 214. Guide 213 further includes a distal end 213b secured to first layer 212. Distal end 213b may be releasably or fixedly secured to second layer 214.

As shown, fat removal device 200 includes four guides 213, situated on the corners of second layer 214 for slidably positioning needles 222 and first layer 212 relative to second layer 214. However, any suitable number of guides 213 may be included between first and second layers 212, 214 and that first layer 212 may comprise any number of individual sections having any number of needles 222 thereon. Thus, the individual sections of first layer 212 may be individually inserted into the target tissue of a patient. The needles 222 on each section may be independently activated. In this manner, the different portions of tissue corresponding to the different sections of first layer 212 may be energized at different intensities and may be energized for different lengths of time to more efficiently perform the procedure.

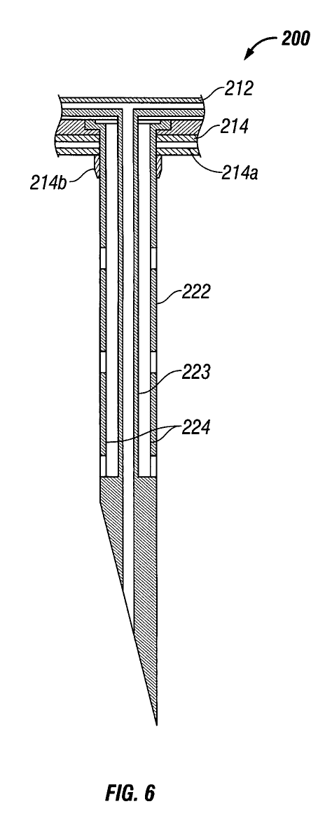

Turning now to FIG. 6, second layer 214 may include a channel or cavity 214a. Cavity 214a is configured for circulating a cooling fluid through second layer 214. In this manner, second layer 214 is prevented from overheating and damaging any skin it may come into contact with. Cavity 214a may be closed, or may instead be connected to fluid source 20 (FIG. 1) or an alternate fluid source. First layer 212 may also include a cavity for circulating a cooling fluid. Second layer 214 further includes tabs or extensions 214b extending distally from second layer 214 about needles 220. Extensions 214b act to insulate the skin of a patent from being affected by the electrosurgical energy being supplied through needle 222.

Still referring to FIG. 6, needle 222 is substantially similar to needle 122 and will only be described in reference to the difference therebetween. Needle 222 includes a first lumen 223 and a second concentric lumen 224. First and second lumens 223, 224 are coupled to one or more fluid/aspirations sources as described above. First and second lumens 223, 224 may deliver the same or different fluids to the target tissue. Alternatively, first or second lumen 223, 224 may be configured for aspiration while the other lumen, 223, 224 may be configured for aspiration. In another configuration, both first and second lumens 223, 224 may be configured for aspiration.

Various changes in form, detail and operation of the fat removal devices of the present disclosure may be made without departing from the spirit and scope of the present disclosure.

What is claimed is:

1. A device for removing and/or sculpting tissue, comprising:
a substantially planar pad; and
an array of needles extending distally from the pad, each of the needles configured for insertion into body tissue, wherein each of the needles is configured to couple to a source of electrosurgical energy.

2. The device of claim 1, wherein each of the needles defines at least one lumen configured for fluid communication with an aspiration/suction source.

3. The device of claim 1, wherein the pad includes a first layer and a second layer.

4. The device of claim 3, wherein the first layer is configured to retain the needles.

5. The device of claim 3, wherein the first layer may be extended relative to the second layer.

6. The device of claim 1, wherein each of the needles may be configured for independent activation.

7. The device of claim 2, wherein each of the needles includes a first lumen configured for suction and a second lumen configured for irrigating fluid.

8. The device of claim 2, wherein the array of needles are configured for simultaneous activation and the at least one lumen is configured for suction.

9. A method of removing and/or sculpting fat, comprising the steps of:
providing a fat removal device having an array of needles extending distally from a pad, each of the needles coupled to a source of electrosurgical energy;
aligning the fat removal device with a target tissue;
penetrating the array of needles into the target tissue; and
activating the source of electrosurgical energy to deliver electrosurgical energy through the array of needles.

10. The method of claim 9, further comprising removing fat from the target tissue via an aspiration/fluid source coupled to the needles.

11. The method of claim 9, further comprising providing fluid through the needles.

12. The method of claim 9, further comprising providing a first power level of electrosurgical energy to a first set of the array of needles and a second power level of electrosurgical energy to a second set of the array of needles.

13. The method of claim 9, further comprising independently activating at least some of the needles.

14. A system for removing and/or sculpting tissue, comprising:
a fat removal device including:
a substantially planar pad; and
an array of needles extending distally from within the pad, each of the needles configured for insertion into body tissue, each of the needles defining at least one lumen therein;
a source of electrosurgical energy coupled to the fat removal device; and
an aspiration/suction source in fluid communication with the at least one lumen.
